(19) United States
(12) Patent Application Publication
(10) Pub. No.: US 2003/0087947 A1
(43) Pub. Date: May 8, 2003

(54) Novel photosensitizers of 9-hydroxypheophorbide-a derivatives used for photodynamic therapy

(76) Inventors: Jeong Woo Cho, Kwangju (KR); Eun Ah Shin, Ulsan (KR); Chang Ho Chung, Kwangju (KR); Young Je Joo, Taejeon (KR); Kyong Joo Na, Taejeon (KR); Pill Soon Song, Kwangju (KR); Phil Sang Chung, Chunan-shi (JP); Mahn Joon Ha, Suwon-shi (JP)

Correspondence Address: Oliff & Berridge, PO Box 19928, Alexandria, VA, 22320, US

(21) Appl. No.: 10/169,771

(22) PCT Filed: Jun. 13, 2001
(86) PCT No.: PCT/KR01/01016

(30) Foreign Application Priority Data
Nov. 20, 2000 (KR) 2000/68864

(51) Int. Cl. C07D 487/22; A61K 31/409; A61N 1/30

(52) U.S. Cl. 514/410; 540/145; 604/20

(57) ABSTRACT

The present invention relates to a 9-hydroxypheophorbide-a derivative, pharmaceutically acceptable salt or acid addition salt used for photodynamic therapy represented by following formula (I) wherein R₁ and R₂ is each independently hydrogen, C1˜C6 linear or branched alkyl, or C3˜C8 aryl.

### TECHNICAL FIELD

The present invention relates to the novel photosensitizers of 9-hydroxypheophorbide-a derivatives used for photodynamic therapy and preparation methods thereof.

### BACKGROUND ART

Recently, the photodynamic therapy is regarded as one of promising cancer therapies. The mechanism of photodynamic therapy is based upon the fact that the singlet oxygen or the free radical which is produced by the chemical reactions among the oxygen in the body destroys the cancer cells or malignant tissues, when the light (photon) is supplied outside together with photosensitizer which is sensitive to photon.

More specifically, in photodynamic therapy, singlet oxygen, oxygen radicals, superoxides or peroxides can be chemically produced by in situ photosensitization in order to destroy the cancer cells or malignant tissues, when visible light (photon) is irradiated to the photosensitizer.

The technique utilizes non-toxic drugs in combination with non-hazardous photosensitizing irradiation, and has the potential of being .more selective yet no less destructive when compared with the commonly used chemotherapy or radiotherapy, and therefore it is expected to increase the quality of life of the treated patients.

Such photodynamic therapy has been researched since 1980's. From 1990's, clinical treatment of photodynamic therapy has been admitted in many countries, such as, Canada, Germany, Japan and United States. Especially, FDA approved this therapy to esophago-cancer in January 1996 and also approved it to the first stage of lung cancer in September 1997. The recent statistics shows that 3,000 cases of this therapy were treated in 1996.

However, the photodynamic therapy has its fundamental problems as follows. i) In case of large volume cancer, this therapy is not available because the light cannot penetrate whole malignant tissues. ii) The cost of photosensitizer is so high. iii) Photosensitizer is slowly metabolized in human body which shows the toxicity. iv) The concentration of photosensitizer in malignant tissues can not be so high, which causes the reduction of therapeutic effect.

The commercially marketed photosensitizer “Photofrin™” approved by FDA in 1996 shows good therapeutic effect and stability. However, after administration of such photosensitizer, it still remains for 5˜6 weeks, which causes the side effect. Further, 630 nm (the maximum light absorption of Photofrin) which is lower light spectrum compared to the optimal light spectrum (650˜850 nm) for photodynamic therapy causes the decline of the penetration of light to the malignant tissues. On the other hand, it has some difficulties for preparing pure Photofrin in chemical synthesis. Therefore, there is a demand for developing the more effective photosensitizer (Chemistry & Industry, pp 739-743, September 1998; Chemical & Engineering News, pp 22-27, November 1998).

On the other hand, as next generation photosensitizer, porphyrins, chlorins, bacteriochlorins and porphycenes have been developed (J. Org. Chem, 63, pp 1646-1656, 1998).

Among them, pheophytins which is prepared after removing metal ion from chlorophylls in the plant shows the good absorption of the long wavelength compared to Photofrin. Further, it can be prepared in a high purity formulation.

Even though the orignal structure of pheophytins prepared after removing metal ion from chlorophylls in the plant can be used as photosensitizer, it can be developed as an excellent photosensitizer after deformation of molecular structure. For example, 10-hydroxypheophytin-a, which is obtained by the oxidation of 10-carbon in the cyclic structure of pheophytin-a, is considered as excellent photosensitizer (Journal of Natural Products, 55, pp 1241-1251, 1992).

10-hydroxypheophytin-a is a compound isolated from the excretion of silkworm having excellent photochemical properties as well as short duration property in the body. Further, it can be produced in a large scale and in a high purity by the oxidation of pheophytin-a. However, it has handicaps due to the fatal toxicity when it is used in a more than certain amount (PCT Publication WO 93/112114).

In order to develop excellent photosensitizer, following conditions have to be satisfied.

i) high photo reaction yield from triplet oxygen to singlet oxygen ii) high absorption of the spectrum more than 650 nm wavelength; iii) high selectivity to the malignant tissues; iv) high excretion from the human body after administration; v) minimum side effect and toxicity; and vi) low cost and mass production in high purity.

As a next generation photosensitizer, chlorophyll and bacteriochlorophyll derivatives was disclosed in U.S. Pat. No. 5,650,292. Especially, pheophytin-a, which is prepared after removing metal ion from chlorophyll, and pheophorbide-a which is prepared after hydrolysis of phytyl radical from pheophytin-a have been considered as excellent photosensitizer, which solves the defects of “Photofrin”. Further, such compounds can be developed by deformation of molecular structure.

However, in the case of pheophorbide-a, there is a drawback of decomposing it slowly in the room temperature. Therefore, this compound shows low stability (Photochemistry and Photobiology, 64, pp 194-204, 1996). Such low stability is the fatal defect as a photosensitizer and the development of new pheophorbide-a derivative has been performed (Tetrahedron Letters, 38, pp 3335-3338, 1997).

To overcome such defect, chlorophyll has been regarded as new photosensitizer. Chlorophyll can be isolated from natural product, especially, blue-green algae. Further, pheophytin can be obtained by the extraction with organic solvent followed by the acid treatment of chlorophyll.

The photosensitizer having chlorophyll structure shows low hydrophilic property, which requires the long excretion time. Therefore, new photosensitizer has been required to overcome long excretion time by deforming the pheophytin structure by the introduction of hydrophilic radical.

In the present invention, the inventors developed an excellent photosensitizer having high selectivity to malignant tissues; easy excretion from the body; low toxicity; and absorption of long wavelength compared to 10-hydroxypheophytin-a, which is considered as good photosensitizer until now.

### DISCLOSURE OF INVENTION

The object of the present invention is to provide a 9-hydroxypheophorbide-a derivative, pharmaceutically acceptable salt or acid addition salt used for photodynamic therapy represented by following formula (I)

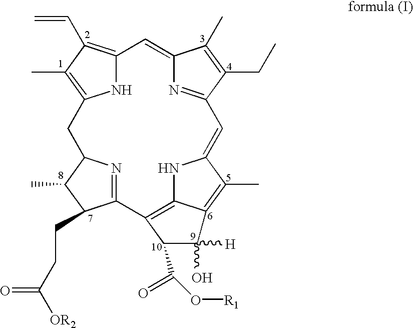

wherein, R₁ and R₂ is each independently hydrogen, C1˜C6 linear or branched alkyl, or C3˜C8 aryl.

The preferred compound among compounds of formula (I) is a compound having methyl as R₁ and hydrogen as R₂.

The compound of formula (I) can be prepared by following methods comprising the steps of:

i) preparing pheophytin-a followed by acid treatment of chlorophyll in blue-green algae; ii) isolating pheophytin-a by silica-gel column chromatography after extraction using organic solvent; iii) reducing from carbonyl group at C9 in pheophytin-a to hydroxy group; iv) hydrolyzing and removing phytyl ester residue; and v) isolating and obtaining the compound of formula (I) using silica-gel column chromatography and thin layer chromatography.

The obtained compound is confirmed by NMR spectrum and high resolution FAB mass.

The further object of the present invention is to provide a cancer treating method using compound of formula (I) as photosensitizer comprising) conjugating compound of formula (I) with liposome; ii) directly inserting said conjugated compound into the cancer tissue; iii) inserting diode laser (660 nm) fiber to the cancer tissue; and iv) treating the cancer tissue by irradiation of light having the wavelength 650˜670 nm.

### BRIEF DESCRIPTION OF DRAWINGS

FIG. 1 is a photograph showing cell cycle regulation after treating 9-hydroxypheophorbide-a of formula (I) to the cancer cell line. It shows that cyclin B1 controls mitosis in the cell is rapidly declined.

FIG. 2 is a diagram showing FACS after treating 9-hydroxypheophorbide-a of formula (I) to the cancer cell line. It shows that the cell cycle arrest occurs in G2/M cell cycle.

FIG. 3 is a photograph showing that 9-HPbD-a and liposome conjugate is administered into the tumor, and that the change of tumor is observed with irradiation of 660 nm diode laser using diffuser tip.

FIG. 4 is a photograph showing that cancer cell line SNU-1041 is implanted into the tissues of nude mouse.

FIG. 5 is a photograph showing that tumor growth causes the death of nude mouth after implantation of cancer cell line SNU-1041.

FIG. 6 is a photograph showing that the initiation of photodynamic therapy is performed after 2 weeks from implantation of cancer cell line SNU-1041.

FIG. 7 is a photograph showing that the tumor is treated by administration of 9-HPbD-a with 660 nm diode laser irradiation.

FIG. 8 is a diagram for relative tumor growth (RTG) analysis to each experimental group.

[symbol] : laser treatment after administration of 9-HPbD-a

[symbol] : laser treatment after administration of Photogem

[symbol] : only administration of 9-HPbD-a

[symbol] : only administration of Photogem

[symbol] : only laser treatment

[symbol] : control

### BEST MODE FOR CARRYING OUT THE INVENTION

In this specification, compound of formula (I) is called as 9-hydroxypheophorbide-a.

9-hydroxypheophorbide-a can be prepared by following methods comprising the steps of i) preparing pheophytin-a followed by acid treatment of chlorophyll in blue-green algae; ii) isolating pheophytin-a by silica-gel column chromatography after extraction using organic solvent; iii) reducing from carbonyl group at C9 in pheophytin-a to hydroxy group; iv) hydrolyzing and removing phytyl ester residue; and v) isolating and obtaining 9-hydroxypheophorbide-a using silica-gel column chromatography and thin layer chromatography.

Further, the present invention provide a cancer treating method using 9-hydroxypheophorbide-a as photosensitizer comprising i) conjugating 9-hydroxypheophorbide-a with liposome; ii) directly inserting said conjugated compound into the cancer tissue; iii) inserting diode laser (660 nm) fiber to the cancer tissue; and iv) treating the cancer tissue by irradiation of light having the wavelength 650˜670 nm.

9-hydroxypheophorbide-a of the present invention has the properties of i) high photo reaction yield from triplet oxygen to singlet oxygen; ii) high absorption of the spectrum more than 650 nm wavelength; iii) high selectivity to the malignant tissues; iv) high excretion from the human body after administration; v) minimum side effect and toxicity; and vi) low cost and mass production in high purity.

9-hydroxypheophorbide-a can be used as pharmaceutical composition, which is useful to several types of cancer including melanoma tumor, brain, ovarian, breast, skin, lung, esophagus and bladder cancer.

The present invention will be more specifically explained by the following examples. However, it should be understood that the examples are intended to illustrate but not in any manner to limit the scope of the present invention.

### EXAMPLE 1

Extraction, Semi-Synthesis and Isolation of 9-hydroxypheophorbide-a

The preparation method was performed in the dark room having red lamp. 20 ml of pyridine-methanol mixed solution (1:1, v/v) was laid on 50 ml of round bottom reactor equipped with nitrogen supplying apparatus, thermometer and dropping funnel. Then, 500 mg of pheophytin-a was dissolved in this solution after removal of metal ion followed by extraction of chlorophyll. 250 mg of NaBH₄ was dissolved in 20 ml of pyridine-methanol mixed solution, and said mixture was added for 1 hour to the reactor through dropping funnel.

The end point of reaction was determined at the time of disappearing pheophytin-a by measuring using HPLC. The reaction time spent from 1.5 hours to 2.5 hours. After cooling the reaction mixture using ice water, 100 ml of HCI solution (2N) was added slowly. Using 100 ml of methylene chloride, the reaction mixture was extracted. Then, HCl was removed using distilled water in the methylene chloride layer.

The water in the methylene chloride layer was removed using sodium sulfate. Then, using rotary evaporator, methylene chloride was removed. The obtained 9-hydroxypheophorbide-a was isolated using silica-gel column chromatography (230˜400 mesh; CH₂Cl₂/acetone=10/1) and thin layer chromatography (CH₂Cl₂/acetone=5/1).

Identification of 9-hydroxypheophorbide-a

The obtained compound was analysed by ¹H and ¹³C NMR. The NMR spectrum showed that phytyl radical was hydrolyzed and converted into carboxyl acid, and that carbonyl group in C9 was converted into hydroxy group. Then, obtained compound was identified as 9-hydroxypheophorbide-a. The molecular weight of obtained compound was 617.2764 (theoretical value: C₃₅H₃₈N₄O₅+Na=617.2740).

### EXAMPLE 2

Cancer Cell Line

Plain melanoma cell line of pharynx cancer SNU-1041 and lung cancer A549 cell line were tissue-cultured. For efficacy comparison, same amount of known photosensitizer “10-hydroxypheophytin-a” and “Photogem” were used.

In-vitro Photodynamic Therapy

The cell line added with “Photogem” was treated and irradiated by red light (570˜650 nm, 0.35 mW/cm², 315 J/m²) from fluorescent lamp and the test sample was treated and irradiated by same red light and laser (660 nm, 0.35 mW/cm², 315 J/m²). After irradiation treatment, the cell line was cultured by adding new medium and 10% of fetal calf serum at 37° C. The cytotoxicity was measured by clonogenic assay after 8 days.

The dose-response curve was prepared after following treatment; i) treating each 25 g/ml of Photogem, 10-hydroxypheophytin-a and 9-hydroxypheophorbide-a (“9-HPbD-a”) to each cell line; ii) laying them for 2 hours; and iii) treating and irradiating the 660 nm light (315 J/m²). To the Photogem sample, the light (630±10 nm) was used by 1 KW Xenon lamp (Model A 5000; Photon Technology International Inc.; Power density 75 mW/cm²) equipped with 5 mm diameter of liquid light guide (2000A, Luminex, Munich, Germany).

Result

Table 1 showed the cytotoxicity of 9-HPbD-a and dose-response result of 9-HPbD-a to the lung cancer A549 cell line by 20 joule of total light dose. In order to minimize the side light reaction by natural light and lamp, the cell distribution and medium exchange were carried out in green light (500 nm), where the experimental samples were not reacted. After 75 μg/ml of reagent treatment on each 10 dishes at the dark culture room, the experimental samples were cultured for 48 hours. Then, the cell death rate was measured. The data was calculated as a mean value after 5 times experiments. Even though 9-HPbD-a showed slightly high cytotoxicity compared to 10-hydroxypheophytin-a, very high dose-response was detected at very low concentration. Therefore, in case of 9-HPbD-a, only 1/100 concentration of known photosensitizer concentration showed the considerable cell death effect. Further, the cytotoxicity of 9-HPbD-a could be neglectable.

TABLE 1
Cytotoxicity and efficacy of photosensitizer under total light
dose (20 joule)

| | | Efficacy | | |
|---|---|---|---|---|
| | Cytotoxicity | (Total light dose: 20 joule) | | |
| Photosensitizer | (75 μg/ml) | 75 | 7.5 | 0.75 μg/ml |
| 10-hydroxypheophytin-a | 3.94% | <70% | 5˜10% | 0% |
| 9-HPbD-a | 10.05% | 100% | 100% | 10˜20% |

As shown in Table 1, each photosensitizer having the concentrations 75, 7.5, 0.75 μg/ml was added to the same cell line and 665 nm of monochromatic light was irradiated in total light dose of 20 joule. 9-HPbD-a showed excellent cell death effect compared to 10-hydroxypheophytin-a. Further, in a small amount (0.75 μg/ml), high cell death effect was detected. Using another synthesized compound of 9-HPbD-a in the other batch, the experiment was performed according to same protocol again. We detected the same result through the repeated experiments.

Table 2 showed the efficacy to the same cell lines using same photosensitizers except that total light dose increased to 100 joule. With a very small amount 0.0185 μg/ml of administration, the cell death treated with 9-HPbD-a occurred according to the increase of total light dose. 9-HPbD-a showed the excellent efficacy in 100 joule irradiation as well as 20 joule irradiation.

TABLE 2
Efficacy of photosensitizer under total light dose (100 joule)

| | Efficacy | | | | |
|---|---|---|---|---|---|
| | (Total light dose: 100 joule) | | | | |
| Photosensitizer | 7.5 μg/ml | 0.75 | 0.075 | 0.0375 | 0.0185 |
| 10-hydroxy- | 100% | 5.85% | 0% | 0% | 0% |
| pheophytin-a | | | | | |
| 9-HPbD-a | ″ | 100% | 100% | 25% | 10% |

### EXAMPLE 3

The change of cell cycle is a key element for evaluating cancer treatment. In most anti-cancer agents, the growth of cancer cells is suppressed by regulating the expression of cell cycle regulation gene. In this example, the change of cell cycle was measured using 9-HPbD-a during the photodynamic therapy. Using various cencentrations of photosensitizer (75˜0.0185 μg/ml) under 20 joule in total light dose, Western blot analysis was carried out to detect the change of cell cycle in accordance with change of regulation activity of cell cycle regulation gene.

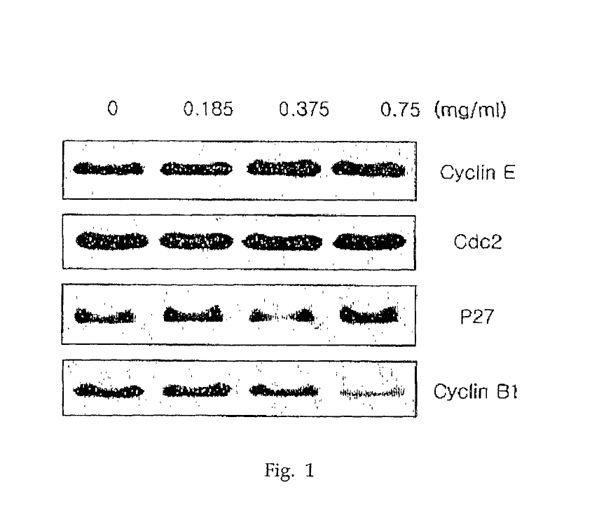

FIG. 1 showed that the amount of cyclin B1 to be involved in mitosis in the cell was rapidly declined. It revealed that 9-HPbD-a affected the regulation of cell cycle.

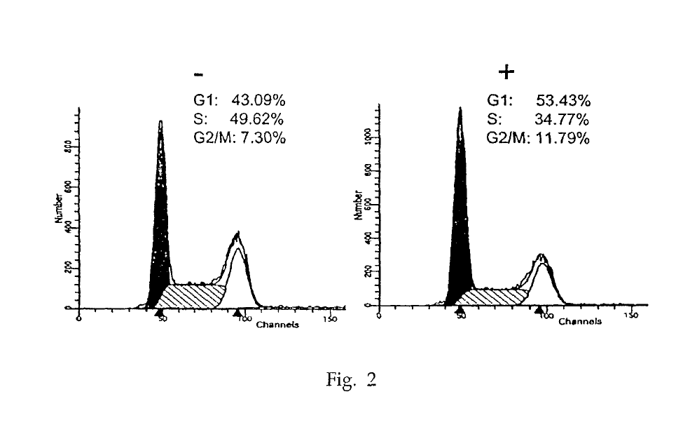

FIG. 2 showed that cell cycle arrest occurred in G2/M cell cycle as shown in FACS analysis in case of using 9-HPbD-a.

### EXAMPLE 4

Implant of Experimental Animals and Cell Lines

As experimental animals, 6 weeks aged male nude mice BALB/C/nu/nu were employed, supplying sterilized water and sterilized feed. These animals were bred in filter-top cage under proper temperature and moisture. To implant cultured cancer cell lines into nude mouse, 0.1 ml of SNU-1041 cell line suspension (10⁷ cells/0.1 ml) was injected subcutaneously. After injection, the tumor occurred and the volume of tumor became increased after more than 8 weeks observation.

Plain melanoma cell line SNU-1041 was implanted into nude mouse. After the lapse of 8 weeks, it was observed that the tumor became spread in all body. The photodynamic therapy was performed only after the tumor volume became 100˜600 mm³.

The experimental groups were divided into 3 groups for photodynamic therapy. 660 nm diode laser was inserted and irradiated using bare fiber tip. By monitoring the temperature outside of the tumor, the therapeutic effect caused by hyperthermia was removed. The optimal irradiation strength and time were measured.

The Therapeutic Effect at the Implanted Tumor

The photodynamic therapy was performed when the volume of implanted tumor became about 100˜600 mm³. The experimental groups were divided into 3 groups.

a. Group 1 (n=10)

0.1˜0.2 ml of 9-HPbD-a (0.75 mg/ml) was injected into tumor using 30G syringe. After 1˜4 hours, 660 nm diode laser was irradiated using 600 m fiber optic tip. 2˜4 places of 5 mm diameter tumor was irradiated using 1 watt continuous wave for 10 minutes.

b. Group 2 (n=10)

0.1˜0.2 ml of Photogem (1 mg/ml) was injected into tumor using 30G syringe. After 1˜4 hours, the irradiation was performed as the same manner of Group 1.

c. Group 3 (n=5)

Without inserting photosensitizer, laser was irradiated as the same manner of Group 1.

As the result of experiment, in Group 1, 3 cases among 10 cases showed complete elimination of tumor and 7 cases showed partial elimination of tumor. In Group 2, 2 cases among 10 cases showed complete elimination of tumor and 8 cases showed partial elimination of tumor. In Group 3, 2 cases among 5 cases showed partial elimination of tumor.

Photodynamic Therapy Using 9-HPbD-a

The experimental groups were divided into 6 groups when the volume of implanted tumor became about 100˜600 mm³. Following treatment was performed and the change of tumor was observed.

a. Group 1 (n=10)

Without any treatment, the change of tumor was observed.

b. Group 2 (n=20)

Without administration of photosensitizer, 660 nm diode laser was irradiated using diffuser tip. Then, the change of tumor was observed.

c. Group 3 (n=20)

With only 0.1˜0.15 ml of administration of 9-HPbD-a and liposome conjugate into the tumor, the change of tumor was observed without irradiation.

d. Group 4 (n=20)

With only 0.1˜0.15 ml of administration of Photogem into the tumor, the change of tumor was observed without irradiation.

e. Group 5 (n=100)

0.1˜0.15 ml of Photogem was administered into the tumor. The change of tumor was observed with irradiation of 660 nm diode laser using diffuser tip after the lapse of 1 hour, 4 hours and 24 hours from administration of Photogem.

f. Group 6 (n=100)

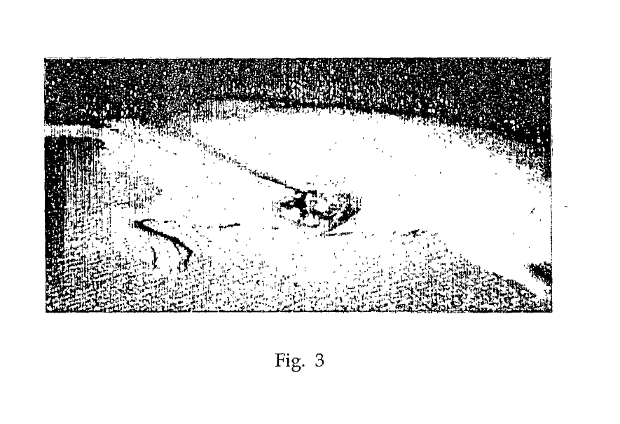

0.1˜0.15 ml of 9-HPbD-a and liposome conjugate was administered into the tumor. The change of tumor was observed with irradiation of 660 nm diode laser using diffuser tip after the lapse of 1 hour, 4 hours and 24 hours from administration of Photogem (FIG. 3).

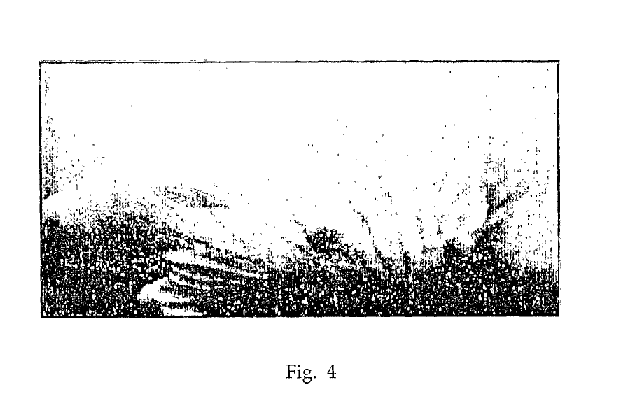

The observed therapeutic effect was effective in all animals over cell level. Such fact was confirmed by using nude mouse experimental system. For experimental system, 6 weeks aged male nude mice BALB/C/nu/nu were employed and bred. Cultured cell line was implanted to the hypodermic tissue of nude mouse (FIG. 4).

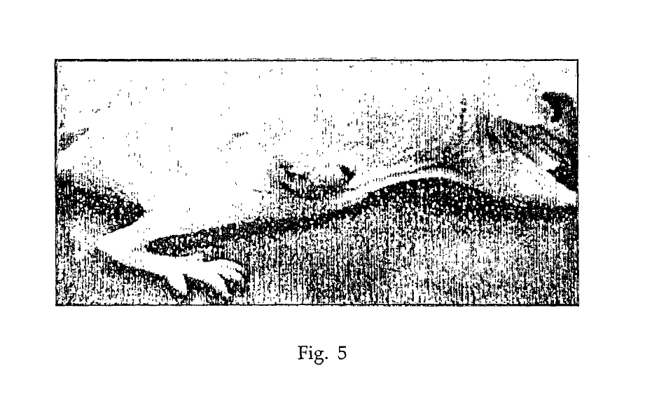

After 1 week from subcutaneous injection of cancer cell line suspension, the growth of tumor was observed. Then, tumor was spread into all body tissues, which caused the death of experimental animals (FIG. 5).

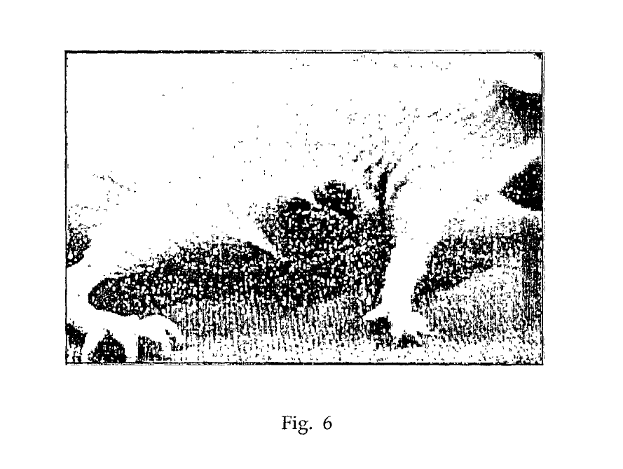

The photodynamic therapy was initiated after 2 weeks from implantation of cancer cell line, when the volume of tumor became about 100˜600 mm³ (FIG. 6).

The measurements of tumor volume were carried out at the time of just before administration of test material; just after administration of 9-HPbD-a; 1 week after administration; 2 weeks after administration; 3 weeks after administration; and 4 weeks after administration. The anti-cancer effect was evaluated by the volume of tumor. The calculation of volume was carried out by using following equation.

V=[4/3×A×B×C]×1/2

(V=volume, A=long axis, B=short axis, C=height)

There was no observation of decline of tumor volume with only inserting Photogem or 9-HPbD-a (Group 3 and 4). In case that 660 nm diode laser was irradiated without administration of photosensitizer (Group 2), the temporary regulation of tumor was observed. However, the tumor grew again within 1 week. Therefore, only the laser irradiation could not treat the tumor.

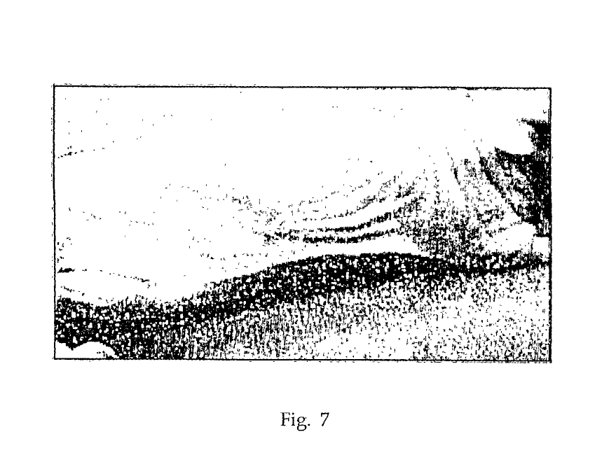

The evaluation of anti-cancer effect was decided after 4 weeks observation of tumor volume. After measuring the relative tumor growth (RTG), 9-HPbD-a was proved to be an excellent photosensitizer for photodynamic therapy compared to 10-hydroxypheophytin-a and Photogem. FIG. 7 is a photograph showing that the tumor is treated by administration of 9-HPbD-a with 660 nm diode laser irradiation.

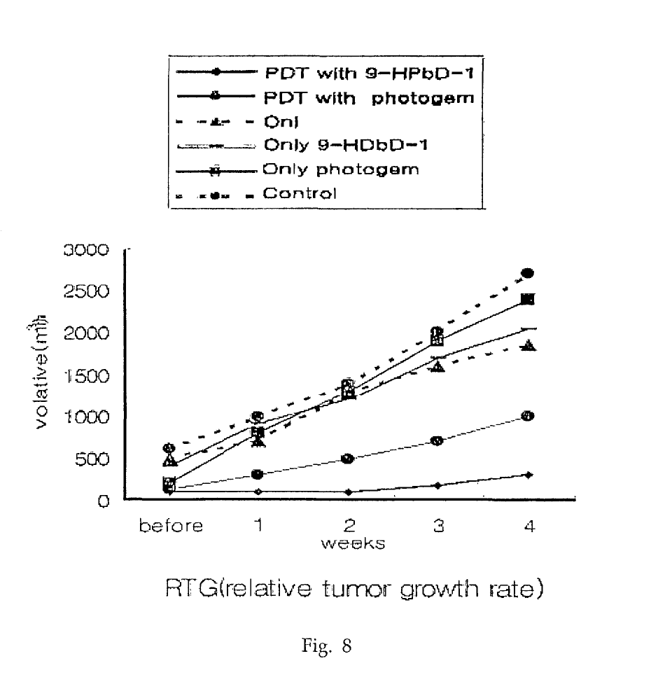

In case of 9-HPbD-a, the volume of tumor was 646 mm³ after 4 weeks from treatment and RTG was 3.5. In case of Photogem, the volume of tumor was 838 mm³ after 4 weeks from treatment and RTG was 6.9 (FIG. 8). Therefore, we could observe the statistically considerable difference between experimental group and control group.

As a conclusion, 9-HPbD-a was proved as an excellent photosensitizer having high selectivity to malignant tissues; easy excretion from the body; low toxicity; and absorption of long wavelength compared to 10-hydroxypheophytin-a, which is considered as good photosensitizer until now.

What is claimed is:

1. A 9-hydroxypheophorbide-a derivative, pharmaceutically acceptable salt or acid addition salt used for photodynamic therapy represented by following formula (I) 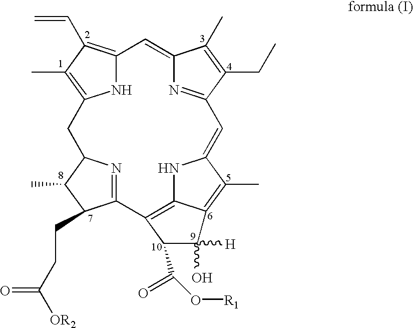
wherein, R₁ and R₂ is each independently hydrogen, C1˜C6 linear or branched alkyl, or C3˜C8 aryl.

2. The 9-hydroxypheophorbide-a derivative, pharmaceutically acceptable salt or acid addition salt used for photodynamic therapy according to claim 1, wherein the preferred compound is a compound having methyl as R₁ and hydrogen as R₂.

3. A process for preparing the compound of formula (I) in claim 1 comprising the steps of:
i) preparing pheophytin-a followed by acid treatment of chlorophyll in blue-green algae;
ii) isolating pheophytin-a by silica-gel column chromatography after extraction using organic solvent;
iii) reducing from carbonyl group at C9 in pheophytin-a to hydroxy group;
iv) hydrolyzing and removing phytyl ester residue; and
v) isolating and obtaining the compound of formula (I) using silica-gel column chromatography and thin layer chromatography.

4. A cancer treating method using compound of formula (I) as photosensitizer comprising:
i) conjugating compound of formula (I) with liposome;
ii) directly inserting said conjugated compound into the cancer tissue;
iii) inserting diode laser (660 nm) fiber to the cancer tissue; and
iv) treating the cancer tissue by irradiation of light having the wavelength 650˜670 nm.
